(19) United States
(12) Patent Application Publication
(10) Pub. No.: US 2024/0237979 A1
(43) Pub. Date: Jul. 18, 2024

(54) FORCEPS AND COMPONENTS THEREFORE

(71) Applicants: ASIF K. MUSTAFA, Willowbrook, IL (US); Devang J. Joshi, Carol Stream, IL (US)

(72) Inventors: ASIF K. MUSTAFA, Willowbrook, IL (US); Devang J. Joshi, Carol Stream, IL (US)

(21) Appl. No.: 18/096,526

(22) Filed: Jan. 12, 2023

(51) Int. Cl. A61B 17/062; A61B 17/06; A61B 17/52

(52) U.S. Cl. CPC A61B 17/0625; A61B 17/06066; A61B 17/52

(57) ABSTRACT

This invention relates to tissue and needle forceps. More specifically, this invention relates to tissue and needle forceps, and components therefore, that utilize a magnetic force to retrieve or manipulate a suture needle and optionally possess an improved user grip and dexterity. The improved forceps comprises a housing located on at least one elongated member of the pair or elongated members proximal to at least one tip of the opposing tips and a magnet affixed to the housing, with the magnet defining both a resting surface configured for operable engagement with the suturing needle and a magnetic pull force to attract the needle to the resting surface. In another embodiment, a magnet is affixed to a housing for removable attachment with an elongated member of the forceps comprising a resting surface defined by the magnet and configured for operable engagement with a suturing needle of a suturing needle and thread assembly, and having a predetermined magnetic pull force defined by the magnet to attract the needle to the resting surface.

### CROSS REFERENCE TO RELATED APPLICATIONS

None

### TECHNICAL FIELD OF THE INVENTION

This invention relates to tissue and needle forceps. More specifically, this invention relates to tissue and needle forceps, and components therefore, that utilize a magnetic force to retrieve or manipulate a suture needle and optionally possess an improved user grip and dexterity.

### BACKGROUND OF THE INVENTION

In applications such as medicine, surgical sutures, also known as stitches, are used to approximate tissue during surgery and/or following an injury. The application generally involves using a needle with an attached length of thread. Numerous types of sutures are utilized, which typically differ by needle shape and size, as well as by thread material; depending upon the characteristic and location of the wound or the specific body tissues being approximated.

A suturing needle is typically utilized as part of a needle and thread assembly, which may comprise an “eyed” needle and thread assembly (i.e., that having a hole or “eye” located opposite of the needle's point) for accepting a separate suture material or thread there-though (similar to common sewing or embroidering needles), or a swaged needle assembly, which is “eyeless” and has a pre-attached and predetermined length of suturing material or thread affixed at an end to a “suture end” of the needle located opposite of its point. The primary advantage of an “eyed” needle and thread assembly is that any thread and needle combination may be assembled to suit a given medical application at hand. The primary advantage of a swaged needle assembly is that the suture end of the needle is narrower than the needle's body, thus minimizing or eliminating the frictional drag of tissue typically exerted on “eyed” needles at the thread attachment site (which typically has a knot protruding from an outer surface of the needle's body). Swaged needle assemblies also avoid the precarious and time-consuming procedure of having to thread the needle itself and tie the two components together, as is required in “eyed” needle and thread assemblies. Thus, due to its foregoing advantages, swaged needle assemblies are preferred over “eyed” needle and thread assemblies and thus are most commonly used in medical practice.

Needles are available in a wide variety of shapes, geometries (i.e., needle point shapes) and sizes. Needle shapes include: straight; half curved or ski; ¼, ⅜, ½, and ⅝ circle; and compound curve. Needle geometries include: taper (needle body is round and tapers smoothly to a point); cutting (needle body is triangular and has a sharpened cutting edge on the inside); reverse cutting (cutting edge on the outside); trocar point or taper-cut (needle body is round and tapered, but ends in a small triangular cutting point); blunt point for sewing friable (i.e., easily torn) tissues; and side cutting or spatula point (flat on top and bottom with a cutting edge along the front to one side) for eye surgery. Needle sizes include: thin bodied needles (used for cardiovascular, ocular, gastroenterological and pediatric surgery); medium bodied needles (used for general tissue closure) and heavy bodied needles (used in certain veterinary applications).

Sutures are typically placed by grasping a needle and thread assembly with a needle driver or needle forceps, which is used by medical professionals to grip the needle and push the assembly through tissue during wound closure, re-anastomosis or other surgical procedures. The forceps may comprise a pair of elongated members defining opposing open and closed ends. The open end defines a pair of opposing tips biased to create a displacement there-between, with the elongated members manipulated by a user's thumb and at least one finger to remove the displacement and create a co-acting pressure between the tips. More specifically, the forceps are commonly held in a “pen grip” between the thumb and index finger (sometimes also the middle finger), with the closed end resting on or proximal to the web-space defined between the user's thumb and index finger. Spring tension, typically generated by a resilient property of the elongated members at the closed end, holds the tips of the open end apart until pressure is applied to the elongated members by the user's digits to close the tips and create a gripping pressure there-between. The gripping pressure of the tips allows a user to easily grasp, manipulate and quickly release needles, sutures and tissue with the forceps' co-acting tips.

Using a forceps during a suturing process, the needle of a needle and thread assembly is initially grasped along its body between the forceps' co-acting tips, and thereafter pressed into a first location of the tissue. The needle is advanced by the forceps along the trajectory of the needle's straight or curved geometry until it emerges from a second location of the tissue. With the needle's point protruding from the tissue at the second tissue location, the forceps releases the needle from between its tips at the first tissue location and thereafter grasps the needle between its tips at the second tissue location to retrieve it. Now again grasping the needle at the second tissue location, the needle and thread assembly is pulled through the tissue, leaving a portion of the thread within the tissue between the first and second locations to create the suture. The foregoing process is repeated as necessary until complete tissue approximation or wound closure occurs, as understood in the art.

Various forceps are present in the medical prior art, to include “Debakey” forceps, which is a type of forceps used in vascular procedures to grasp retrieve (i.e., pull) suturing needles and avoid tissue damage during manipulation. Debakey or vascular forceps typically possess an extended length (i.e. up to 36 cm long), to enable use within a chest cavity or other vascular area, and have a coarsely ribbed grip to enable a grasping of the tool in the presence of blood or other bodily fluids. However, numerous disadvantages are associated with prior art vascular forceps and other forceps utilized during intricate surgical procedures on minute and/or delicate tissues. For example, grasping and pulling the small and delicate thin-bodied needles commonly utilized in such surgical procedures may lead to a blunting of the needle's tip and/or a bending or distortion of the needle's body, thus both making the suturing process difficult and causing possible significant tissue damage. Such tissue damage can lead to potentially life-threatening bleeding, especially when suturing blood vessels. After the needle is damaged, the suture may need to be cut out (i.e., with swaged needles) and the process restarted with a fresh suture; adding more time and cost to the procedure as well as additional tissue trauma resulting from re-suturing of the same tissue.

Also, retrieving needles with forceps is often difficult given precarious tissue and/or needle angles. For example, if the curved trajectory of a curved needle is not properly followed during the retrieval process, significant tissue damage may occur via a tearing of the tissue by the needle. Furthermore, once retrieved, re-grasping the needle between the tips of a forceps at a given angle, necessary to accommodate the geometry of the needle itself and/or the angle of the tissue to be sutured, is often difficult because the needle may have an incorrect angle of presentation to the tips and/or move when grasped between the tips; both resulting in an incorrect position of the needle within the forceps. This leads to the wasting of precious operative time where time if of the essence for the wellbeing of the patient, with users often using their fingers to reposition the needle, thus exposing them to needle sticks and associated blood-borne health risks.

Further disadvantages are associated with prior art forceps in relation to their grasp and manipulation. Despite the presence of ribbing or other textured surfaces defined on the gripping surfaces of the forceps, they nonetheless slip within the operator's hand due to the presence of blood and/or other slippery fluids. A slipping of the forceps within the hand disrupts the intricate suturing procedure, possibly resulting in needle and/or tissue damage and the associated waste of valuable operative time addressing it.

Thus, what is needed are forceps that retrieve and manipulate small and delicate thin-bodied needles without the risk of blunting, bending, distorting or otherwise damaging the needles themselves. The forceps should have the ability to retrieve and draw needles comfortably despite variation in tissue angle and needle geometry. The forceps should further have the ability to reapply needles onto the driver at appropriate angles for subsequent needle insertions without needle slip. Moreover, they should not slip when gripped within the operator's hand due to the presence of blood and other slippery fluids. The present invention satisfies the foregoing needs while presenting other advantages over the prior art as well.

### SUMMARY OF THE INVENTION

This invention relates to tissue and needle forceps. More specifically, this invention relates to tissue and needle forceps, and components therefore, that utilize a magnetic force to retrieve or manipulate a suture needle and optionally possess an improved user grip and dexterity.

In one embodiment, a forceps of the type comprising a pair of elongated members defining opposing open and closed ends is utilized, with the open end defining a pair of opposing tips that are biased to create a displacement between the tips. The closed end is defined by ends of the elongated members that are located opposite the tips and are unitary with one another or permanently affixed to one another. As is understood in the art, the elongated members of the forceps are manipulated by a user's digits, preferably a thumb and at least one finger, to open and/or close (i.e., displace and/or remove the displacement of) the opposing tips.

The bias creating the displacement between the opposing tips is preferably generated by an elastic deformation inherent of the material property of the elongated members existing at the closed end. More specifically, the material property of the elongated members allow them to undergo elastic deformation such that, when the digits of a user force the displaced opposing tips towards one another by applying opposing forces to the members, the members and tips return to their original shape and displaced position upon a removal of the forces. However, it is understood that a spring (i.e., a torsion, coil of leaf spring, not shown) may be operably engaged with the elongated members at the closed end to bias the tips away from one another as well.

As an improvement to the forceps, a magnet is located on at least one member of the pair of elongated members, proximal to the respective tip(s), with the magnet defining both a resting surface configured for operable engagement with a suturing needle of a needle and thread assembly, and a predetermined magnetic pull force to attract the needle to the resting surface. In one embodiment where the magnet is affixed to a housing, the resting surface is inwardly curved for operable engagement with a curve of an outer surface of the needle. The curve defined by the magnet's resting surface approximates the curve of the outer surface defined over the length of the suturing needle such that the curve of the resting surface, when oriented about parallel to the length of needle, allows the needle to securely rest against the resting surface when drawn into the magnet by the magnetic pull force. A mounting surface of the magnet is defined opposite of the resting surface and abuts an interface surface of the housing, with the mounting surface outwardly curved and the interface surface inwardly curved to accept an abutment against one another. Although the remainder of the housing inwardly of the interface surface defines a “C-shape,” it is understood that the remainder of the housing can define other geometrical shapes as well, to include various polygons and triangles and other arcuate shapes.

In another embodiment, the resting surface of the magnet is outwardly-facing and affixed within the housing, with the housing again located on at least one elongated member. More specifically, the resting surface is located proximal to an interior base of a hollow cup defined by each housing and extending from each elongated member proximal to the respective tips. The interior base of each hollow cup is located proximal to each respective elongated member and defines the housing's interface surface, with each hollow cup also defining an outer open end. The mounting surface of the magnet, again located opposite of the magnet's resting surface, again abuts the interface surface of the housing, with the mounting and interface surfaces both preferably defining planes to accept an abutment against one another.

The hollow cup further defines a cylindrical inner surface, extending outwardly from the base, that transitions into a frusto-conical inner surface terminating at the cup's outer open end and configured to accept a forward end of the needle therein such that the forward end is drawn inwardly into the cup by the magnetic pull force of the magnet, thus allowing the forward needle end to securely rest against the magnet's resting surface within the cup. The frusto-conical inner surface of the cup acts to “funnel” or otherwise guide the forward needle end into the cylindrical inner surface and towards the resting surface of the magnet. A guiding of the needle into the cup via the frusto-conical inner surface is critical in facilitating a time-efficient introduction of the needle end therein during time-sensitive surgical procedures. In one embodiment, the magnetic pull force of the magnet is at least equal to a greater of the frictional force exerted on the suturing needle and thread assembly by a bodily tissue and the weight of the suturing needle and thread assembly itself.

The above-recited housings defining the respective “C-shape” and hollow cup are each affixed to or defined by the mount defining a sleeve configured for frictional engagement with the at least one elongated member, with the sleeve defining a through opening terminating in upper and lower sleeve ends and having an internal circumferal contact surface extending there-between. The through opening of the sleeve is configured to accept an insertion of the at least one elongated member there-through, with the opening defining an area that is less than an area defined by a cross-section of the elongated member(s).

The internal circumferal contact surface of the sleeve is configured for frictional contact with an outer surface of the member(s), with the sleeve preferably comprised of a resilient, elastomeric material that elastically stretches around the member inserted there-through and conforms to the shape of the member(s) to create a tight, elastomeric friction fit there-between. The mount defining the sleeve may optionally comprise one of a plurality of selectively interchangeable mounts of varying through opening diameter configured to accept the insertion of the at least one member there-through. The mount defining the sleeve may also optionally comprise one of a plurality of selectively interchangeable mounts of common or varying though opening diameter and having a variety of housing geometries (i.e., “C-shaped” or cup) and/or dimensions, and/or a variety of associated magnet geometries, dimensions and/or pulling forces.

In further embodiments, the housings defining the respective “C-shape” and hollow cup are each affixed to or defined by the mount defining a band configured for mechanical engagement with the at least one elongated member. The band defines a through opening terminating in upper and lower band ends and having an internal circumferal contact surface extending there-between, and configured to accept an insertion of the at least one elongated member there-through, with the opening defining a predetermined geometrical shape and size to accommodate a variety of elongated members.

The mount defining the band may optionally comprise one of a plurality of selectively interchangeable mounts of varying geometry configured to accept the insertion of the at least one member there-through. The mount defining the band may also optionally comprise one of a plurality of selectively interchangeable mounts of common or varying geometry and having a variety of housing geometries (i.e., “C-shaped” or cup) and/or dimensions, and/or a variety of associated magnet geometries, dimensions and/or pulling forces.

At least one threaded, through bore is defined through the band to accept the insertion of a threaded fastener, such as a screw, bolt or allen screw there-through, with an inner end of the fastener configured for interfering engagement with the outer surface of the member(s). Thus, with the at least one member inserted though the through opening of the band, the threaded fastener is rotated clockwise within the threaded bore until its inner end interferingly contacts the outer surface of the member(s), and at least a portion of both the band's circumferential inner contact surface and the outer surface of the member(s) become interferingly engaged to secure the band and at least one member to one another. In the these embodiments, the band is preferably comprised of a rigid material capable of accommodating the force of the threaded fastener engaging the at least one elongated member. The housing is preferably unitary with the sleeve band such that it also comprises the rigid material of the mount, while each magnet is preferably permanently affixed to each housing.

In additional embodiments, the housings defining the respective “C-shape” and hollow cup are each affixed to or defined by a mount defining an open collar configured for resistive engagement with the at least one elongated member. The collar defines a through opening terminating in upper and lower collar ends and having a partial internal circumferal contact surface extending there-between to define a lateral opening. The collar is configured to accept an axial insertion of the at least one elongated member through the through opening or a lateral insertion of the member(s) through the lateral opening. The collar's through opening defines a predetermined geometrical shape and size to accommodate the cross-sectional shape and thickness of a given elongated member.

Thus, the through opening may define a polygon, circle, oval, a portion of any shape, as well combinations of them as well to conform to a like geometry of the elongated member(s). Regardless of shape and size of the through opening, it defines an approximate area that is less than the area defined by a cross-section of the at least one elongated member such that the collar can elastically deform about the at least one member such that the collar(s) and member(s) can establish a resistance fit with one another. The mount defining the collar may optionally comprise one of a plurality of selectively interchangeable mounts of varying geometry and/or opening area configured to accept the insertion of one of a variety of the at least one member and/or of like geometry there-through. The mount defining the collar may also optionally comprise one of a plurality of selectively interchangeable mounts of common or varying geometry and having a variety of housing geometries (i.e., “C-shaped” or cup) and/or dimensions, and/or a variety of associated magnet geometries, dimensions and/or pulling forces.

The open collar is comprised of materials understood in the art as providing an elastically deformable property. As such, a gripping bias is created by the collar about the at least one member generated by the elastic deformability inherent of the material property of the collar. More specifically, the material property of the open collar allows the collar to undergo elastic deformation such that, when the at least one member is inserted through the collar's through of lateral openings, the collar elastically expands around the member(s) such that the circumferential collar's contact surface and the outer surface of the member(s) become resistively engaged to secure the collar and member(s) to one another. The housing is preferably unitary with the collar such that it also comprises the rigid material of the mount, while each magnet is preferably permanently affixed to each housing via the use of a strong adhesive, such as that comprising an epoxy or other similar adhesive understood in the art. However, it is understood that the housing may be affixed to the collar with bonding means or mechanical fasteners understood in the art, with the housing comprising materials common to or different from the mount.

As a further improvement to the forceps, a support is optionally located on at least one member of the pair of members, proximal to the closed end, with the support configured for operable engagement with at least one digit of the user. In a preferred embodiment, the support comprises a ring configured to accept an insertion of a thumb there-through. However, it is understood that the ring may be unitary with the at least one member.

In alternate embodiments, the support is removably attachable with the at least one elongated member, proximal to the closed end, with the support affixed to or defined by a stay defining a sleeve configured for frictional engagement with the elongated member. The sleeve defines a through opening terminating in upper and lower sleeve ends and having an internal circumferal contact surface extending there-between. The through opening of the sleeve is configured to accept an insertion of the at least one elongated member there-through, with the opening defining an area that is less than an area defined by a cross-section of the elongated member(s).

The internal circumferal contact surface of the sleeve is configured for frictional contact with an outer surface of the member(s), with the sleeve preferably comprised of a resilient, elastomeric material that elastically stretches around the member inserted there-through and conforms to the shape of the member(s) to create a tight, elastomeric friction fit there-between. The stay defining the sleeve may optionally comprise one of a plurality of selectively interchangeable stays of varying opening diameter configured to accept the insertion of the at least one member there-through. The stay defining the sleeve may also optionally comprise one of a plurality of selectively interchangeable stays of common or varying diameter and having a variety of support geometries and/or dimensions.

The support is preferably unitary with the sleeve such that it also comprises the resilient, elastomeric material of the stay. However, it is understood that the support may be affixed to the sleeve with bonding means or mechanical fasteners understood in the art, with the support comprising materials common with, or different from the stay. In one embodiment, the support is affixed to or defined by a stay defining a band configured for mechanical engagement with the at least one elongated member. The band preferably defines a through opening terminating in upper and lower band ends and having an internal circumferal contact surface extending there-between, and configured to accept an insertion of the at least one elongated member there-through, with the opening defining a predetermined geometrical shape and size to accommodate a variety of elongated members of similar geometry. The stay defining the band may optionally comprise one of a plurality of selectively interchangeable stays of varying size and geometry configured to accept the insertion of the at least one member of similar size and geometry there-through, and may also optionally comprise one of a plurality of selectively interchangeable stays of common or varying geometry and having a variety of support geometries and/or dimensions.

At least one threaded, through bore is defined through the band to accept the insertion of a threaded fastener, such as a screw, bolt or allen screw there-through, with an inner end of the fastener configured for interfering engagement with the outer surface of the member(s). Thus, with the at least one member inserted though the through opening of the band, the threaded fastener is rotated clockwise within the threaded bore until its inner end interferingly contacts the outer surface of the at least one elongated member and at least a portion of both the circumferential contact surface and the member's outer surface become interferingly engaged to secure the band and member(s) to one another. In these embodiments, the band is preferably comprised of a rigid material capable of accommodating the force of the threaded fastener engaging the at least one elongated member. The support is preferably unitary with the band such that it also comprises the rigid material of the mount stay. However, it is understood that the support may be affixed to the band with bonding means or mechanical fasteners understood in the art, with the support comprising materials common with or different from the stay.

The support is affixed to or defined by a stay defining an open collar configured for resistive engagement with the at least one elongated member, with the collar defining a through opening terminating in upper and lower collar ends and having a partial internal circumferal contact surface extending there-between to define a lateral opening. The collar is configured to accept an axial insertion of the at least one elongated member there-through or a lateral insertion of the member(s) through the lateral opening, with the collar's through opening defining a predetermined geometrical shape and size to accommodate a cross-sectional shape and thickness of a given elongated member of similar size and geometry. Regardless of size and geometry, the through opening of the open collar defines an approximate area that is less than the area defined by a cross-section of the at least one elongated member such that the collar can elastically deform about the at least one member such that the collar(s) and member(s) can establish a resistance fit with one another. The stay defining the collar may optionally comprise one of a plurality of selectively interchangeable stays of varying geometry and opening area configured to accept the insertion of the at least one member of similar geometry and size there-through, with the stay also optionally comprising one of a plurality of selectively interchangeable stays of common or varying geometry and opening area having a variety of support geometries and/or dimensions.

The open collar is comprised of materials understood in the art as providing an elastically resilient property. As such, a gripping bias is created by the collar about the at least one member generated by the elastic deformability inherent of the material property of the collar. More specifically, the material property of the open collar allows the collar to undergo elastic deformation such that, when the at least one member is inserted through the collar's through or lateral openings, the collar elastically expands around the member(s) such that the collar's circumferential contact surface and outer surface of the member(s) become restively engaged to secure the collar and member(s) to one another. The support is preferably unitary with the collar such that it also comprises the rigid material of the stay. However, it is understood that the support may be affixed to the collar with bonding means or mechanical fasteners understood in the art, with the support comprising materials common to or different from the stay. The foregoing, removably attachable support and magnet and housing components thus permit a user to interchangeably select these components, alone or in varying combinations with one another, to “build” a custom forceps utilizing any one or more of the components.

While this foregoing description and accompanying figures are illustrative of the present invention, other variations in system and method are possible without departing from the invention's spirit and scope.

### DESCRIPTION OF THE DRAWINGS

FIG. 1 illustrates a perspective view of a typical forceps;

FIG. 2 illustrates a perspective view of the typical forceps of FIG. 1 with an improvement comprising a first embodiment of a needle magnet and housing;

FIG. 3 illustrates a close-up perspective view of the needle magnet and housing of FIG. 2;

FIG. 4 illustrates a perspective view of the typical forceps of FIG. 1 with another improvement comprising a second needle embodiment of a needle magnet and housing;

FIG. 5 illustrates a close-up sectional view of the needle magnet and housing of FIG. 4;

FIG. 6A illustrates a removable first embodiment of the magnet and housing of FIGS. 2 and 3;

FIG. 6B illustrates a removable first embodiment of the magnet and housing of FIGS. 4 and 5;

FIG. 7A illustrates a removable second embodiment of the magnet and housing of FIGS. 2 and 3;

FIG. 7B illustrates a removable second embodiment of the magnet and housing of FIGS. 4 and 5;

FIG. 8A illustrates a removable third embodiment of the magnet and housing of FIGS. 2 and 3;

FIG. 8B illustrates a removable third embodiment of the magnet and housing of FIGS. 4 and 5;

FIG. 9 illustrates a perspective view of the typical forceps of FIG. 1 with another improvement comprising a support;

FIG. 10 illustrates a removable first embodiment of the support of FIG. 9;

FIG. 11 illustrates a removable second embodiment of the support of FIG. 9; and

FIG. 12 illustrates a removable third embodiment of the support of FIG. 9.

### DESCRIPTION OF THE EMBODIMENTS

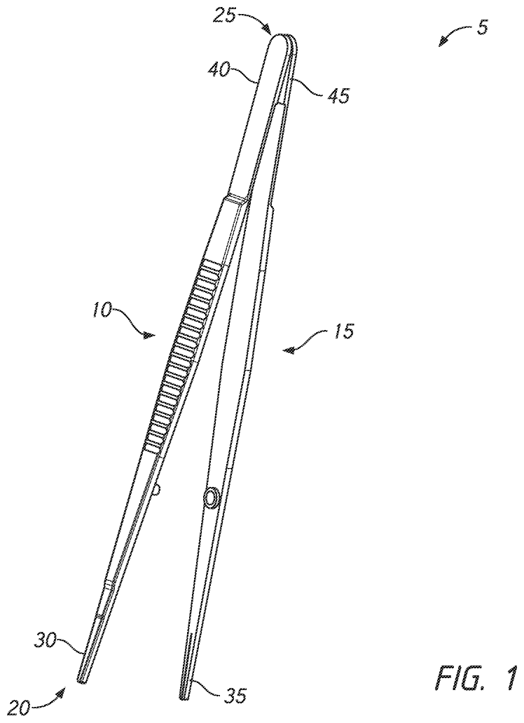

This invention relates to tissue and needle forceps. More specifically, this invention relates to an improved tissue and needle forceps, and components therefore, that utilize a magnetic force to retrieve or manipulate a suture needle and optionally possess an improved user grip and dexterity. FIG. 1 illustrates a forceps 5 of the type comprising a pair of elongated members 10 and 15 defining opposing open and closed ends 20 and 25. The open end 20 defines a pair of opposing tips 30 and 35 that are biased to create a displacement between the tips, while the closed end 25 is defined by ends 40 and 45 of the elongated members 10 and 15 that are located opposite the tips and are unitary with one another or permanently affixed to one another. As understood in the art, the elongated members 10 and 15 of the forceps 5 are manipulated by a user's digits, preferably a thumb and at least one finger, to open and/or close (i.e., displace and/or remove the displacement of) the opposing tips 30 and 35.

In a preferred embodiment of the invention, the elongated members 10 and 15 are comprised of stainless steel. However, it is understood that the elongated members 10 and 15 may be comprised of aluminum, plastic and other similar materials understood in the art as providing elastic and resilient properties. As such, the bias creating the displacement between the opposing tips 30 and 35 is preferably generated by an elastic deformation inherent of the material property of the elongated members 10 and 15 existing at the closed end 25. More specifically, the material property of the elongated members 10 and 15 allow them to undergo elastic deformation such that, when the digits of a user force the displaced opposing tips towards one another by applying opposing forces to the members, the members and tips return to their original shape and displaced position upon a removal of the forces. However, it is understood that a spring (i.e., a torsion, coil of leaf spring, not shown) may be operably engaged with the elongated members 10 and 15 at the closed end 25 to bias the tips 30 and 35 away from one another as well.

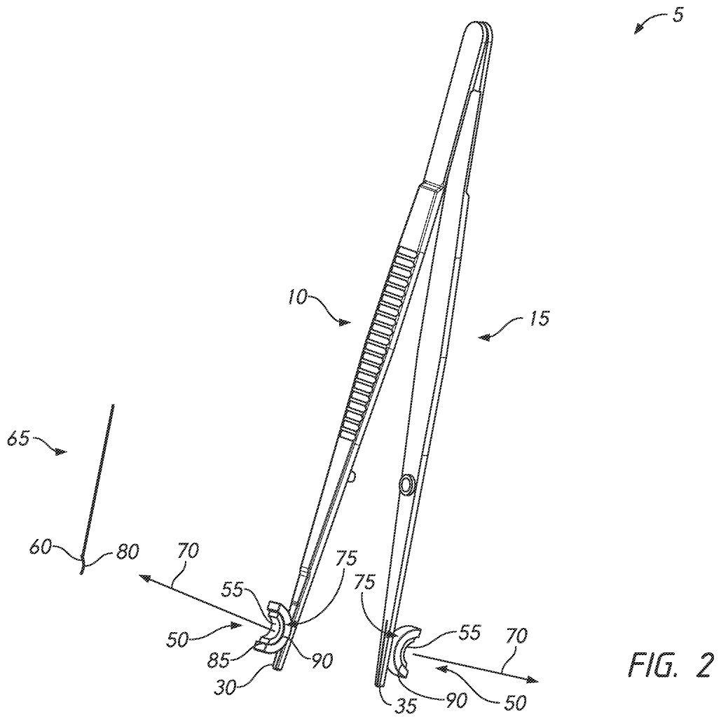

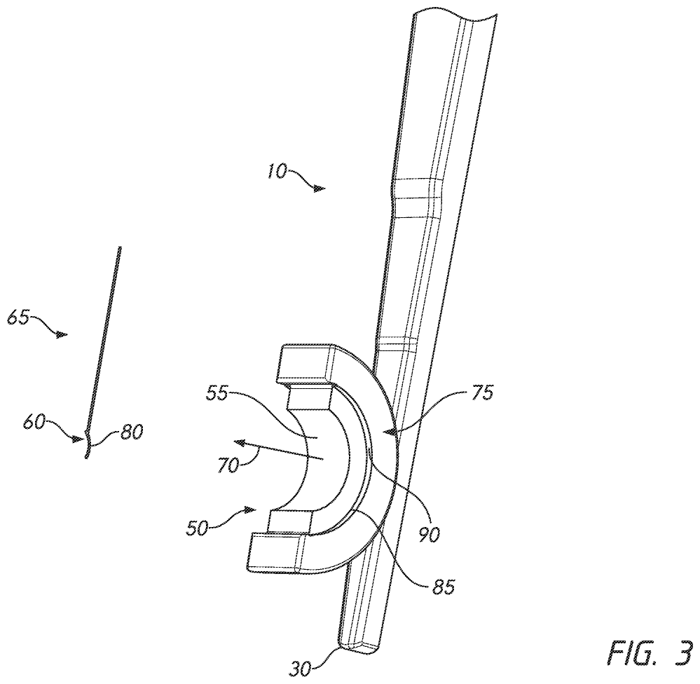

Referring to FIGS. 2, 3, 4 and 5 as an improvement to the forceps 5, a magnet 50 is located on at least one member of the pair of elongated members 10 and 15, proximal to the respective tip(s) 30 or 35, with the magnet defining both a resting surface 55 configured for operable engagement with a suturing needle 60 of a needle and thread assembly 65, and a predetermined magnetic pull force 70, to be further discussed, to attract the needle to the resting surface. In the embodiment of FIGS. 2 and 3, the magnet 50 is affixed to a housing 75, namely, housings located on both elongated members 10 and 15, with the resting surface 55 inwardly curved for operable engagement with a curve of an outer surface 80 of the needle 60. The curve defined by the magnet's resting surface 55 thus approximates the curve of the outer surface 80 defined over the length of the suturing needle 80 such that the curve of the resting surface, when oriented about parallel to the length of needle, allows the needle to securely rest against the resting surface when drawn into the magnet 50 by the magnetic pull force 70. To accommodate the curvatures of variously-sized needles utilized in medical and veterinary applications, the resting surface 55 of the magnet defines a radius of between about 1 mm and 15 mm.

A mounting surface 85 of the magnet 50 is defined opposite of the resting surface 55 and abuts an interface surface 90 of the housing 75, with the mounting surface outwardly curved, and the interface surface inwardly curved to accept an abutment against one another. Although the remainder of the housing 75 inwardly of the interface surface 90 defines a “C-shape,” it is understood that the remainder of the housing can define other geometrical shapes as well, to include various polygons and triangles, as well as other arcuate shapes.

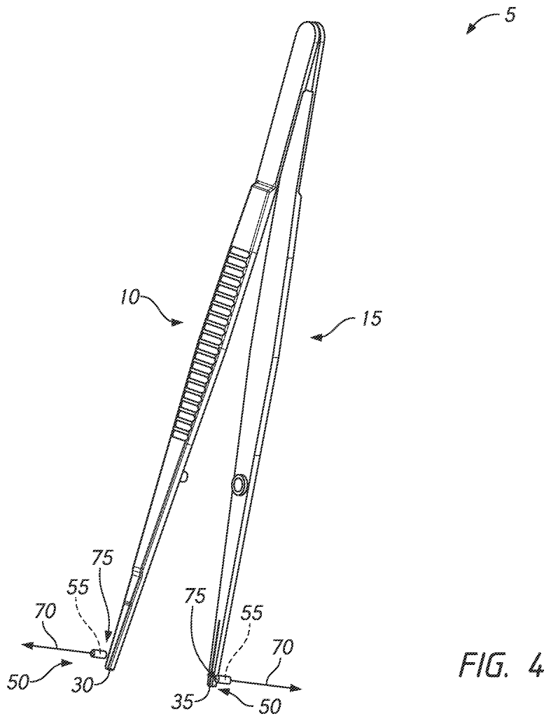

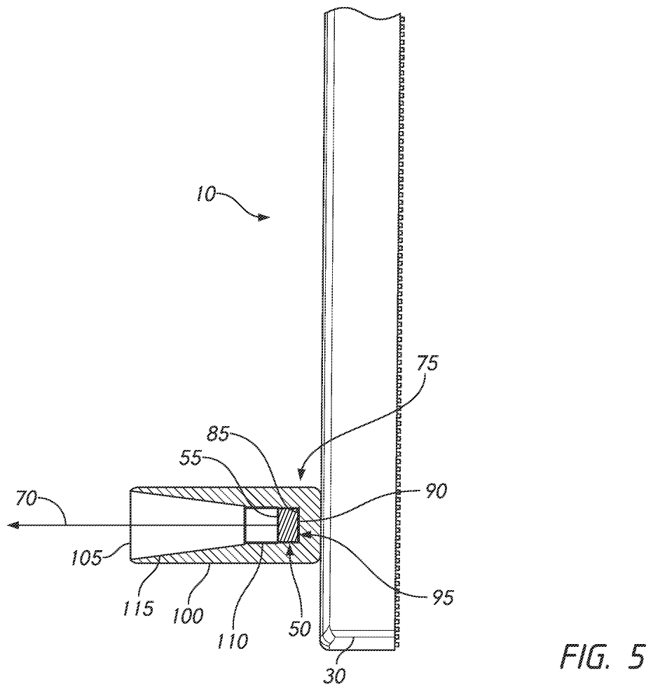

In another embodiment illustrated in FIGS. 4 and 5, the resting surface 55 of the magnet 50 is outwardly-facing and affixed within the housing 75, with the housing again located on each elongated member 10 and 15. More specifically and referring to FIG. 5, the resting surface 55 is located proximal to an interior base 95 of a hollow cup 100 defined by each housing 75 and extending from each elongated member 10 and 15 proximal to the respective tips 30 and 35. The interior base 95 of each hollow cup 100 is located proximal to each respective elongated member and defines the housing's interface surface 90, with each hollow cup also defining an outer open end 105. The mounting surface 85 of the magnet 50, again located opposite of the magnet's resting surface 55, again abuts the interface surface 90 of the housing 75, with the mounting and interface surfaces both preferably defining planes to accept an abutment against one another.

The hollow cup 100 further defines a cylindrical inner surface 110, extending outwardly from the base 96, that transitions into a frusto-conical inner surface 115 terminating at the cup's outer open end 105 and configured to accept a forward end 120 of the needle 60 therein such that the forward end is drawn inwardly into the cup by the magnetic pull force 70 of the magnet 50; thus allowing the forward needle end to securely rest against the magnet's resting surface 55 within the cup. The frusto-conical inner surface 115 of the cup 100 acts to “funnel” or otherwise guide the forward needle end 120 into the cylindrical inner surface 110 and towards the resting surface 55 of the magnet 50. A guiding of the needle 60 into the cup 100 via the frusto-conical inner surface 115 is critical in facilitating a time-efficient introduction of the needle end 120 therein during time-sensitive surgical procedures.

To accommodate the aforementioned variously-sized needles utilized in medical and veterinary applications, the cylindrical inner surface 110 of the cup 100 preferably defines a diameter of between about 0.1 mm and 19 mm, and a depth between its frusto-conical inner surface 115 and resting surface 55 of between about 0.5 mm and 20 mm. The frusto-conical inner surface 115 of the cup 100 preferably defines an introduction diameter that is between 1% and about 10000% larger than the diameter of the cylindrical inner surface 110, and a length of between about 0.1 mm and 30 mm between the outer open end 105 and cylindrical inner surface.

Referring again to FIGS. 2 through 5, in one embodiment, the magnetic pull force 70 of the magnet 50 is at least equal to a greater of the frictional force exerted on the suturing needle and thread assembly 65 by a bodily tissue (not shown) and the weight of the suturing needle and thread assembly itself, with the pull force preferably defining between about 0.01 and 0.1 Tesla; more preferably between about 0.2 and 0.6 Tesla; and optimally about 0.3 Tesla. In another embodiment, the magnet 50 preferably defines a magnetic field underlying the pull force 70 of between about 100 and 10,000 Gauss; more preferably between about 2000 and 6000 Gauss; and optimally about 3000 Gauss.

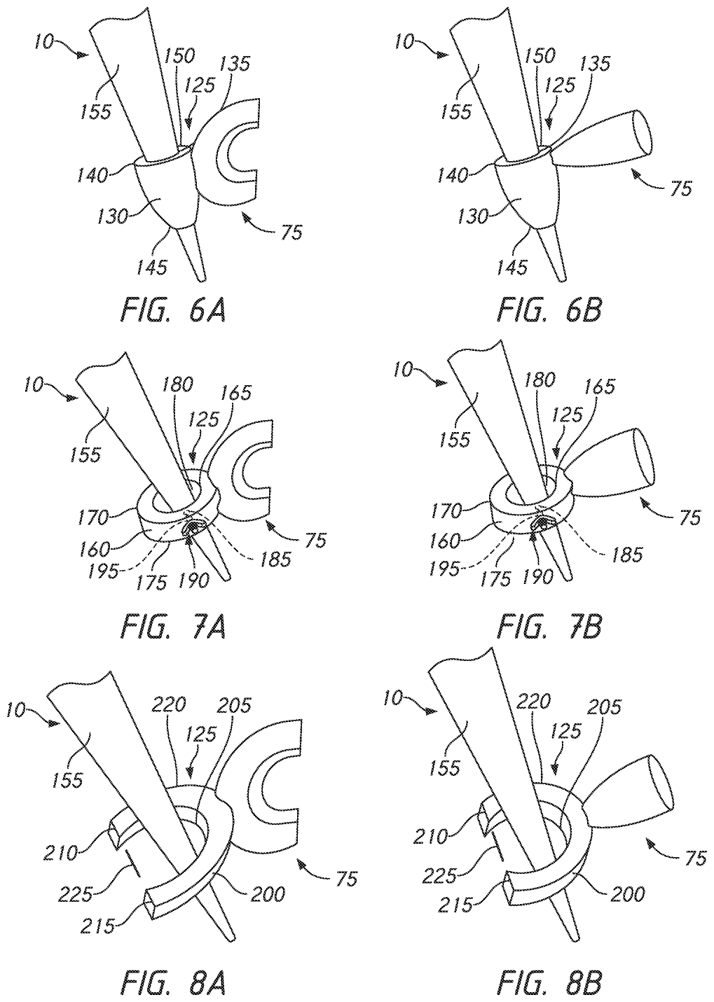

In the embodiments of FIGS. 2 through 5, each magnet 50 is preferably permanently affixed to each housing 75 via the use of a strong adhesive, such as an epoxy or other adhesive understood in the art. However, it is understood that rivets, screws, nuts and bolts and similar mechanical fasteners understood in the art may be utilized as well. Also in the embodiments of FIGS. 2 through 4, each housing 75 is affixed to each member 10 and 15 via the use of rivets, screws, nuts and bolts and similar mechanical fasteners understood in the art. However, it is also understood that braising, soldering, welding, adhesive may be utilized as well. It is further understood that each housing may be unitary with each member. In the alternate embodiments of FIGS. 6A through 8B, each housing 75 is removably attachable to the at least one elongated member 10 and/or 15 via a mount 125 affixed to or defined by the housing. Referring now to FIGS. 6A and 6B, the above-recited housings 75 defining the respective “C-shape” and hollow cup are each affixed to or defined by the mount 125 defining a sleeve 130 configured for frictional engagement with the at least one elongated member 10 and/or 15 (only member 10 is illustrated by example). The sleeve 130 defines a through opening 135 terminating in upper and lower sleeve ends 140 and 145 and having an internal circumferal contact surface 150 extending there-between.

The through opening 135 of the sleeve 130 is configured to accept an insertion of the at least one elongated member 10 and/or 15 there-through, with the opening defining an area that is less than an area defined by a cross-section of the elongated member(s). The internal circumferal contact surface 150 of the sleeve 130 is configured for frictional contact with an outer surface 155 of the member(s), with the sleeve preferably comprised of a resilient, elastomeric material that elastically stretches around the member inserted there-through and conforms to the shape of the member(s) to create a tight, elastomeric friction fit there-between. To accommodate a wide variety of the at least one member 10 and/or 15, the through opening 135 of the sleeve 130 is between about 1 mm and 50 mm in diameter. The mount 125 defining the sleeve 130 may optionally comprise one of a plurality of selectively interchangeable mounts of varying through opening 135 diameter configured to accept the insertion of the at least one member 10 and/or 15 there-through. The mount 125 defining the sleeve 130 may also optionally comprise one of a plurality of selectively interchangeable mounts of common or varying though opening 135 diameter and having a variety of housing 75 geometries (i.e., “C-shaped” or cup) and/or dimensions, and/or a variety of associated magnet 50 geometries, dimensions and/or pulling forces 70.

The housing 75 is preferably unitary with the sleeve 130 such that it also comprises the resilient, elastomeric material of the mount 125, while each magnet 50 is preferably permanently affixed to each housing via the use of a strong adhesive, such as that comprising an epoxy or other similar adhesive understood in the art. However, it is understood that the housing 75 may be affixed to the sleeve 130 with a bonding means or mechanical fasteners understood in the art, with the housing comprising materials common with, or different from the mount 125.

In further embodiments illustrated in FIGS. 7A and 7B, the housings 75 defining the respective “C-shape” and hollow cup are each affixed to or defined by the mount 125 defining a band 160 configured for mechanical engagement with the at least one elongated member 10 and/or 15 (only member 10 is illustrated by example). The band 160 defines a through opening 165 terminating in upper and lower band ends 170 and 175 and having an internal circumferal contact surface 180 extending there-between, and configured to accept an insertion of the at least one elongated member 10 and/or 15 there-through, with the opening defining a predetermined geometrical shape and size to accommodate a variety of elongated members. Thus, the through opening 165 may define a polygon, circle, oval, a portion of any shape, as well combinations of them as well to conform to like geometries of the at least one member 10 and/or 15. The mount 125 defining the band 160 may optionally comprise one of a plurality of selectively interchangeable mounts of varying geometry configured to accept the insertion of the at least one member 10 and/or 15 there-through. The mount 125 defining the band 160 may also optionally comprise one of a plurality of selectively interchangeable mounts of common or varying geometry and having a variety of housing 75 geometries (i.e., “C-shaped” or cup) and/or dimensions, and/or a variety of associated magnet 50 geometries, dimensions and/or pulling forces 70.

At least one threaded, through bore 185 is defined through the band 160 to accept the insertion of a threaded fastener 190, such as a screw, bolt or allen screw there-through, with an inner end 195 of the fastener configured for interfering engagement with the outer surface 155 of the member(s) 10 and/or 15. Thus, with the at least one member 10 and/or 15 inserted though the through opening 165 of the band 160, the threaded fastener 190 is rotated clockwise within the threaded bore 185 until its inner end 195 interferingly contacts the outer surface 155 of the member(s), and at least a portion of both the band's circumferential inner contact surface 180 and the outer surface 155 of the member(s) become interferingly engaged to secure the band and at least one member to one another.

In these embodiments, the band 160 is preferably comprised of a rigid material such as stainless steel, plastic, polymer, aluminum, thermoplastic and similar materials capable of accommodating the force of the threaded fastener 190 engaging the at least one elongated member 10 and/or 15. The housing 75 is preferably unitary with the sleeve band 160 such that it also comprises the rigid material of the mount 125, while each magnet 50 is preferably permanently affixed to each housing via the use of a strong adhesive, such as that comprising an epoxy or other similar adhesive understood in the art. However, it is understood that the housing 75 may be affixed to the band 160 with bonding means or mechanical fasteners understood in the art, with the housing comprising materials common to or different from the mount 125.

In additional embodiments illustrated in FIGS. 8A and 8B, the housings 75 defining the respective “C-shape” and hollow cup are each affixed to or defined by a mount 125 defining an open collar 200 configured for resistive engagement with the at least one elongated member 10 and/or 15 (only member 10 is illustrated by example). The collar 200 defines a through opening 205 terminating in upper and lower collar ends 210 and 215 and having a partial internal circumferal contact surface 220 extending there-between to define a lateral opening 225. The collar 200 is configured to accept an axial insertion of the at least one elongated member 10 and/or 15 through the through opening 205 or a lateral insertion of the member(s) through the lateral opening 225. The collar's through opening 205 defines a predetermined geometrical shape and size to accommodate the cross-sectional shape and thickness of a given elongated member 10 and/or 15.

Thus, the through opening 205 may define a polygon, circle, oval, a portion of any shape, as well combinations of them as well to conform to a like geometry of the elongated member(s). Regardless of shape and size of the through opening 205, it defines an approximate area that is less than the area defined by a cross-section of the at least one elongated member 10 and/or 15 such that the collar 200 can elastically deform about the at least one member such that the collar(s) and member(s) can establish a resistance fit with one another, to be further discussed.

The mount 125 defining the collar 200 may optionally comprise one of a plurality of selectively interchangeable mounts of varying geometry and/or opening area configured to accept the insertion of one of a variety of the at least one member 10 and/or 15 of like geometry there-through. The mount 125 defining the collar may also optionally comprise one of a plurality of selectively interchangeable mounts of common or varying geometry and having a variety of housing 75 geometries (i.e., “C-shaped” or cup) and/or dimensions, and/or a variety of associated magnet 50 geometries, dimensions and/or pulling forces.

The open collar 200 is comprised of stainless steel, aluminum, plastic and other similar materials understood in the art as providing an elastically deformable property. As such, a gripping bias is created by the collar 200 about the at least one member 10 and/or 15 generated by the elastic deformability inherent of the material property of the collar. More specifically, the material property of the open collar 200 allows the collar to undergo elastic deformation such that, when the at least one member 10 and/or 15 is inserted through the collar's through of lateral openings 205 or 225, the collar elastically expands around the member(s) such that the circumferential collar's contact surface 220 and the outer surface 155 of the member(s) become restively engaged to secure the collar and member(s) to one another. The housing 75 is preferably unitary with the collar 200 such that it also comprises the rigid material of the mount 125, while each magnet 50 is preferably permanently affixed to each housing via the use of a strong adhesive, such as that comprising an epoxy or other similar adhesive understood in the art. However, it is understood that the housing 75 may be affixed to the collar 200 with bonding means or mechanical fasteners understood in the art, with the housing comprising materials common to or different from the mount 125.

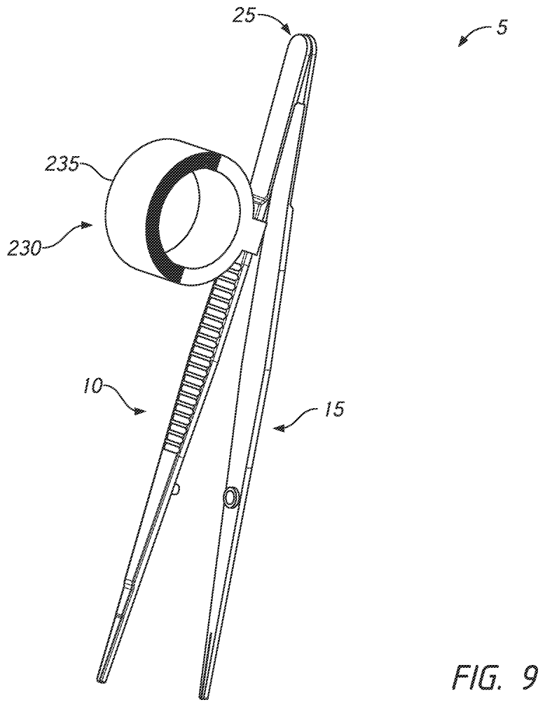

Referring to FIG. 9 as a further improvement to the forceps 5 illustrated in FIG. 9, a support 230 is optionally located on at least one member 10 and/or 15 of the pair of members, proximal to the closed end 25, with the support configured for operable engagement with at least one digit of the user. In a preferred embodiment, the support 230 comprises a ring 235 configured to accept an insertion of a thumb there-through. The ring 235 is affixed to the at least one member 10 and/or 15 via the use of rivets, screws, nuts, bolts and similar mechanical fasteners understood in the art. However, it is also understood that braising, soldering, welding, adhesive may be utilized as well. It is further understood that the ring 235 may be unitary with the at least one member 10 and/or 15. Although FIG. 9 illustrates the forceps 5 improved with the support 230 only, it is understood that the forceps may also include the aforementioned magnet 50 and housing improvements 75 as well.

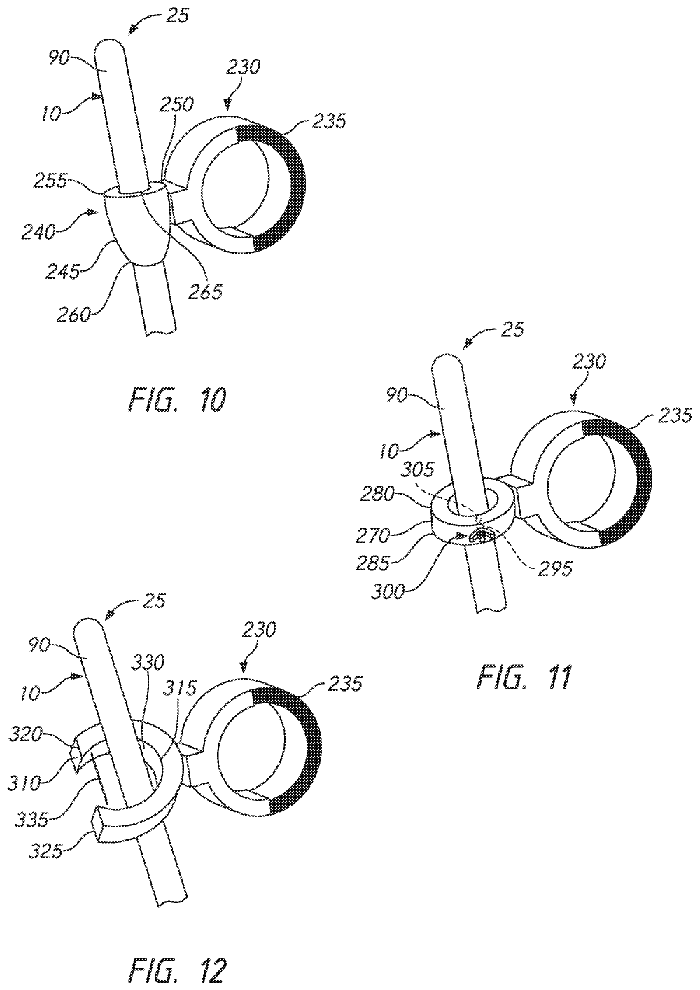

In the alternate embodiments of FIGS. 10 through 12 the support 230, preferably defining a ring 235, is removably attachable with the at least one elongated member 10 and/or 15, proximal to the closed end 25. Referring now to FIG. 10, the support 230 is affixed to or defined by a stay 240 defining a sleeve 245 configured for frictional engagement with the elongated member 10 and/or 15 (only member 10 is illustrated by example). The sleeve 245 defines a through opening 250 terminating in upper and lower sleeve ends 255 and 260 and having an internal circumferal contact surface 265 extending there-between.

The through opening 260 of the sleeve 245 is configured to accept an insertion of the at least one elongated member 10 and/or 15 there-through, with the opening defining an area that is less than an area defined by a cross-section of the elongated member(s). The internal circumferal contact surface 265 of the sleeve 245 is configured for frictional contact with an outer surface 90 of the member(s), with the sleeve preferably comprised of a resilient, elastomeric material that elastically stretches around the member inserted there-through and conforms to the shape of the member(s) to create a tight, elastomeric friction fit there-between. To accommodate a wide variety of the at least one member 10 and/or 15, the through opening 250 of the sleeve 245 is between about 1 and 15 mm in diameter. The stay 240 defining the sleeve 245 may optionally comprise one of a plurality of selectively interchangeable stays of varying opening diameter configured to accept the insertion of the at least one member 10 and/or 15 there-through. The stay 240 defining the sleeve 245 may also optionally comprise one of a plurality of selectively interchangeable stays of common or varying diameter and having a variety of support 230 geometries and/or dimensions.

The support 230 is preferably unitary with the sleeve 245 such that it also comprises the resilient, elastomeric material of the stay 240. However, it is understood that the support 230 may be affixed to the sleeve 245 with bonding means or mechanical fasteners understood in the art, with the support comprising materials common with, or different from the stay 240. As illustrated in FIG. 11, the support 230 is affixed to or defined by a stay 240 defining a band 270 configured for mechanical engagement with the at least one elongated member 10 and/or 15 (only member 10 is illustrated by example). The band 270 defines a through opening 275 terminating in upper and lower band ends 280 and 285 and having an internal circumferal contact surface 290 extending there-between, and configured to accept an insertion of the at least one elongated member 10 and/or 15 there-through, with the opening defining a predetermined geometrical shape and size to accommodate a variety of elongated members of similar geometry. Thus, the through opening 275 may define a polygon, circle, oval, a portion of any shape, as well combinations of them as well. The stay 240 defining the band 270 may optionally comprise one of a plurality of selectively interchangeable stays of varying size and geometry configured to accept the insertion of the at least one member 20 and/or 15 of similar size and geometry there-through. The stay 240 defining the band 270 may also optionally comprise one of a plurality of selectively interchangeable stays of common or varying geometry and having a variety of support 230 geometries and/or dimensions.

At least one threaded, through bore 295 is defined through the band 270 to accept the insertion of a threaded fastener 300, such as a screw, bolt or allen screw there-through, with an inner end 305 of the fastener configured for interfering engagement with the outer surface 90 of the member(s) 10 and/or 15. Thus, with the at least one member 10 and/or 15 inserted though the through opening 275 of the band 270, the threaded fastener 300 is rotated clockwise within the threaded bore 295 until its inner end 305 interferingly contacts the outer surface 90 of the at least one elongated member and at least a portion of both the circumferential contact surface 290 and the member's outer surface 90 become interferingly engaged to secure the band 270 and member(s) to one another. In these embodiments, the band 270 is preferably comprised of a rigid material such as stainless steel, plastic, polymer, aluminum, thermoplastic and similar materials capable of accommodating the force of the threaded fastener 300 engaging the at least one elongated member 10 and/or 14. The support 230 is preferably unitary with the band 270 such that it also comprises the rigid material of the mount stay 240. However, it is understood that the support 230 may be affixed to the band 270 with bonding means or mechanical fasteners understood in the art, with the support comprising materials common with or different from the stay 240.

With reference to FIG. 12, the support 230 is affixed to or defined by a stay 240 defining an open collar 310 configured for resistive engagement with the at least one elongated member 10 and/or 15 (only member 10 is illustrated by example). The collar 310 defines a through opening 315 terminating in upper and lower collar ends 320 and 325 and having a partial internal circumferal contact surface 330 extending there-between to define a lateral opening 335. The collar 310 is configured to accept an axial insertion of the at least one elongated member 10 and/or 15 there-through or a lateral insertion of the member(s) through the lateral opening 335. The collar's through opening 315 defines a predetermined geometrical shape and size to accommodate a cross-sectional shape and thickness of a given elongated member of similar size and geometry. Thus, the through opening 315 may define a polygon, circle, oval, a portion of any shape, as well combinations of them as well. Regardless of size and geometry, the through opening 315 of the open collar 310 defines an approximate area that is less than the area defined by a cross-section of the at least one elongated member 10 and/or 15 such that the collar 310 can elastically deform about the at least one member such that the collar(s) and member(s) can establish a resistance fit with one another, to be further discussed. The stay 240 defining the collar 310 may optionally comprise one of a plurality of selectively interchangeable stays of varying geometry and opening area configured to accept the insertion of the at least one member and/or 15 of similar geometry and size there-through. The stay 240 defining the collar 310 may also optionally comprise one of a plurality of selectively interchangeable stays of common or varying geometry and opening area having a variety of support 230 geometries and/or dimensions.

The open collar 310 is comprised of stainless steel, aluminum, plastic and other similar materials understood in the art as providing an elastically resilient property. As such, a gripping bias is created by the collar 310 about the at least one member 10 and/or 15 generated by the elastic deformability inherent of the material property of the collar. More specifically, the material property of the open collar 310 allows the collar to undergo clastic deformation such that, when the at least one member 10 and/or 15 is inserted through the collar's through or lateral openings 315 Or 335, the collar elastically expands around the member(s) such that the collar's circumferential contact surface 330 and outer surface 90 of the member(s) become restively engaged to secure the collar and member(s) to one another. The support 230 is preferably unitary with the collar 310 such that it also comprises the rigid material of the stay 240. However, it is understood that the support 230 may be affixed to the collar 319 with bonding means or mechanical fasteners understood in the art, with the support comprising materials common to or different from the stay 240. The foregoing, removably attachable support 230 and magnet 50 and housing 75 components thus permit a user to interchangeably select these components, alone or in varying combinations with one another, to “build” a custom forceps utilizing any one or more of the components.

While this foregoing description and accompanying figures are illustrative of the present invention, other variations in system and method are possible without departing from the invention's spirit and scope.

We claim:

1. An improved forceps of the type comprising a pair of elongated members defining opposing open and closed ends, the open ends defining a pair of opposing tips biased to create a displacement between the tips, the elongated members manipulated by a user's thumb and at least one finger to remove the displacement and grasp a suturing needle of a needle and thread assembly between the opposing tips, the improvement comprising:
a housing located on at least one elongated member of the pair or elongated members proximal to at least one tip of the opposing tips;
a magnet affixed to the housing, the magnet defining both a resting surface configured for operable engagement with the suturing needle and a magnetic pull force to attract the needle to the resting surface.

2. The improved forceps of claim 1 wherein the resting surface of the magnet defines an inwardly curved surface configured for operable engagement with the suturing needle.

3. The improved forceps of claim 1 wherein the resting surface of the magnet is located at an interior base of a hollow sleeve defined by the housing and extending from the at least one elongated member, the base located proximal to the at least one elongated member and the sleeve defining an open end configured to accept a forward end of the needle therein.

4. The improved forceps of claim 1 wherein the magnetic pull force is at least equal to a greater of a frictional force exerted on the suturing needle and thread assembly by a bodily tissue and a weight of the suturing needle and thread assembly.

5. The improved forceps of claim 4 wherein the at least one magnet defines a magnetic field of between about 100 and 10,000 Gauss.

6. The improved forceps of claim 1 further comprising a support located on the at least one member of the pair of members, the at least one support configured for operable engagement with at least one digit of the user.

7. The improved forceps of claim 6 wherein the support comprises a ring configured to accept an insertion of the at least one digit there-through.

8. The improved forceps of claim 1 wherein the housing is removably attachable with the at least one member.

9. The improved forceps of claim 1 wherein the housing is unitary with the at least one member.

10. The improved forceps of claim 1 wherein the housing is affixed to the at least one member.

11. The improved forceps of claim 6 wherein the support is removably attachable with the at least one member.

12. The improved forceps of claim 6 wherein the support is unitary with the at least one member.

13. The improved forceps of claim 6 wherein the support is affixed to the at least one member.

14. A magnet affixed to a housing, for removable attachment with an elongated member of a forceps, comprising a resting surface defined by the magnet and configured for operable engagement with a suturing needle of a suturing needle and thread assembly, and a predetermined magnetic pull force defined by the magnet to attract the needle to the resting surface.

15. The magnet of claim 14 wherein the resting surface defines an inwardly curved surface configured for operable engagement with the needle.

16. The magnet of claim 14 wherein the resting surface of the magnet is located at an interior base of a hollow sleeve defined by the housing, the sleeve defining an open end configured to accept a forward end of the needle therein.

17. The magnet of claim 14 wherein the magnetic pull force is at least equal to the greater of a frictional force exerted on the suturing needle and thread assembly by a tissue and a weight of the suturing needle and thread assembly.

18. The magnet of claim 14 wherein the magnet defines a magnetic field of between about 100 and 10,000 Gauss.

19. A support configured for removable attachment to an elongated member of a forceps, the support configured for operable engagement with at least one digit of the user.

20. The support of claim 19 wherein the configuration comprises a ring configured to accept an insertion of the at least one digit there-through.
